(19) United States
(12) Patent Application Publication
(10) Pub. No.: US 2021/0299463 A1
(43) Pub. Date: Sep. 30, 2021

(54) A HEARING LOSS ALLEVIATING DEVICE AND METHOD OF USE OF SAME

(71) Applicant: Helium 3 Resources Pty Ltd, South Perth (AU)

(72) Inventor: Raymond Dennis Palmer, South Perth, WA (US)

(21) Appl. No.: 16/321,455

(22) PCT Filed: Jul. 27, 2017
(86) PCT No.: PCT/AU2017/050776

§ 371 Date: Jan. 28, 2019

(30) Foreign Application Priority Data
Jul. 29, 2016 (AU) 2016902995

(51) Int. Cl. A61N 5/06; A61N 2/00; A61N 2/02

(52) U.S. Cl. CPC A61N 5/0603; A61N 2/002; A61N 2/006; A61N 2/02; A61N 2005/067; A61N 2005/0647; A61N 2005/0653; A61N 2005/0662; A61N 2005/0605

(57) ABSTRACT

A hearing loss alleviating device sized and configured to be received, at least in part, by a central auditory system of a subject and a method of using the same. The hearing loss alleviating device includes a power supply and at least one of an electromagnetic radiation generator, an electromagnetic field generator, and a high frequency sound generator. When powered, the hearing loss alleviating device generates at least one of electromagnetic radiation, an electromagnetic field, and high frequency sound.

### FIELD

The present disclosure relates to a hearing loss alleviating device sized and configured to be received, at least in part, by a central auditory system of a subject, that has a therapeutic application. The present disclosure also relates to methods of using the hearing loss alleviating device.

### BACKGROUND

An estimated 70 million people worldwide are affected by hearing loss to some extent. Hearing loss is a chronic condition the incidence of which is proportionally related to age. It is well-established that a loss of hearing has an adverse impact on the psychological and social well-being of the person affected by hearing loss. Generally, people affected by hearing loss feel isolated and tend to withdraw from social interaction as a means to minimize frustration arising from their inability to hear speech and consequent inability to follow conversations.

Hearing loss can be classified as arising from two aetiologies: conductive and/or sensorineural. These aetiologies may give rise to hearing loss individually or in combination. Conductive hearing loss is a result of an impediment to the normal mechanisms of the outer and/or middle ear. Sensorineural hearing loss, on the other hand, is a consequence of damage to the central auditory system, in particular to the sensory components and/or nerves of the central auditory system. People affected by sensorineural hearing loss typically do not benefit from auditory prostheses that stimulate mechanical perturbation of the cochlear fluid. These people can, however, benefit from a wearable or implantable device that stimulates the central auditory system of the person wearing the device.

### SUMMARY

The following is a broad summary of various exemplary embodiments of a hearing loss alleviating device sized and configured to be received, at least in part, by a central auditory system of a subject, that has a potential therapeutic application. The present disclosure also relates to methods of using the hearing loss alleviating device.

According to an embodiment, there is provided a hearing loss alleviating device sized and configured to be received, at least in part, by a central auditory system of a subject, the device comprising: an electromagnetic radiation generator configured to irradiate at least a part of the central auditory system of the subject; a power supply; and an interface configured to selectably adjust electromagnetic radiation in a wavelength range of 10 nm-1 mm produced by the electromagnetic radiation generator when energized by the power supply.

In another embodiment, the hearing loss alleviating device may comprise an electromagnetic radiation generator controller in communication with the electromagnetic radiation generator. In another embodiment, the hearing loss alleviating device may include an independent energy source in communication with the electromagnetic radiation generator. In another embodiment, the electromagnetic radiation generator may include at least one light emitting diode. In another embodiment, the electromagnetic radiation generator may be a laser. In another embodiment, the electromagnetic radiation generator may be an arc lamp. In another embodiment, the electromagnetic radiation generator may be an incandescent lamp. In another embodiment, the hearing loss alleviating device may comprise a frequency adjuster to vary the wavelength of the electromagnetic radiation being emitted from the electromagnetic radiation generator. In another embodiment, the hearing loss alleviating device may comprise a timing circuit configured to shut down the electromagnetic radiation generator after a predetermined amount of elapsed time.

In another embodiment, the hearing loss alleviating device may comprise a control configured to intensity of the electromagnetic radiation emitted by the electromagnetic radiation generator.

In another embodiment, the hearing loss alleviating device may comprise a conduit, the conduit including: an electromagnetic radiation delivery system in optical communication with the electromagnetic radiation generator; and an objective lens at a distal end of the conduit, the objective lens in communication with a display system.

In another embodiment, there is provided a hearing loss alleviating device sized and configured to be received, at least in part, by a central auditory system of a subject, the device comprising: an electromagnetic field generator configured to produce an electromagnetic field within at least a part of the central auditory system of the subject; a power supply; and an interface configured to selectably adjust the electromagnetic field in a range of 0.001-200 μT produced by the electromagnetic field generator when energized by the power supply.

In another embodiment, the hearing loss alleviating device may include an electromagnetic field generator controller in communication with the electromagnetic field generator. In another embodiment, the hearing loss alleviating device may include an independent energy source in communication with the electromagnetic field generator. In another embodiment, the hearing loss alleviating device may comprise a magnetic field strength adjuster to vary the magnetic field strength of the electromagnetic field being emitted from the electromagnetic field generator. In another embodiment, the hearing loss alleviating device may comprise a timing circuit configured to shut down the electromagnetic field generator after a predetermined amount of elapsed time.

In another embodiment, the hearing loss alleviating device may comprise a conduit, the conduit including: an electromagnetic field delivery system in operative communication with the electromagnetic field generator; and an objective lens at a distal end of the conduit, the objective lens in communication with a display system.

In another embodiment, the objective lens may communicate with the display system via a fibre optic system. In another embodiment, the conduit is a flexible conduit. In another embodiment, the flexible conduit may comprise a steering system for navigating the distal end of the conduit. In another embodiment, the flexible conduit may comprise a channel to allow access of medical instruments. In another embodiment, the hearing loss alleviating device may comprise a speaker. In another embodiment, the speaker may be in electrical communication with the power supply. In another embodiment, the speaker may be configured to electrically communicate with a speaker controller. In another embodiment, the speaker may be housed in a housing and the housing may be sized and configured to be received by an ear canal of a subject.

In another embodiment, the housing may have arranged thereon at least one electromagnetic radiation source. In another embodiment, the at least one electromagnetic radiation source may be in electrical communication with the power supply. In another embodiment, the at least one electromagnetic radiation source may be in operative communication with the electromagnetic radiation generator. In another embodiment, the hearing loss alleviating device may comprise an electromagnetic radiation source controller in electrical communication with the at least one electromagnetic radiation source.

In another embodiment, there is provided a method of treating or ameliorating hearing loss, the hearing loss having an aetiology selected from the group consisting of: (a) impaired blood flow; (b) impaired metabolism; (c) neuronal damage; (d) infection; and any combination of (a) to (d); the method comprising irradiating at least a part of a central auditory system of a subject having hearing loss with electromagnetic radiation having a wavelength in a range of 10 nm-1 mm to stimulate an alleviating effect on the hearing loss of the subject.

In another embodiment, irradiating at least a part of a central auditory system of a subject with electromagnetic radiation having a wavelength in a range of 10 nm-1 mm may invigorate at least a part of an Organ of Corti of the subject. In another embodiment, irradiating at least a part of a central auditory system of a subject with electromagnetic radiation having a wavelength in a range of 10 nm-1 mm may invigorate at least a part of a vestibulocochlear nerve of the subject. In another embodiment, irradiating at least a part of a central auditory system of a subject with electromagnetic radiation having a wavelength in a range of 10 nm-1 mm may increase blood supply to at least a part of a cochlea of the subject.

In another embodiment, there is provided a method of treating or ameliorating hearing loss, the hearing loss having an aetiology selected from the group consisting of: (a) impaired blood flow; (b) impaired metabolism; (c) neuronal damage; (d) infection; and any combination of (a) to (d); the method comprising exposing at least a part of a central auditory system of a subject having hearing loss to an electromagnetic field having an electromagnetic field strength in a range of 0.001-200 μT to stimulate an alleviating effect on the hearing loss of the subject.

In another embodiment, exposing at least a part of a central auditory system of a subject having hearing loss to an electromagnetic field having an electromagnetic field strength in a range of 0.001-200 μT may invigorate at least a part of an Organ of Corti of the subject. In another embodiment, exposing at least a part of a central auditory system of a subject having hearing loss to an electromagnetic field having an electromagnetic field strength in a range of 0.001-200 μT may invigorate at least a part of a vestibulocochlear nerve of the subject. In another embodiment, exposing at least a part of a central auditory system of a subject having hearing loss to an electromagnetic field having an electromagnetic field strength in a range of 0.001-200 μT may increase blood supply to at least a part of a cochlea of the subject.

According to an embodiment, there is provided hearing loss alleviating device sized and configured to be received, at least in part, by a central auditory system of a subject, the device comprising: a power supply; a high frequency sound wave generator configured to emit high frequency sound waves into at least a part of the central auditory system of the subject; and an interface configured to selectably adjust high frequency sound waves in a frequency range of 800,000 Hz-2,000,000 Hz produced by the high frequency sound wave generator when energized by the power supply.

In another embodiment, the hearing loss alleviating device may include a high frequency sound wave generator controller in communication with the high frequency sound wave generator. In another embodiment, the hearing loss alleviating device may include an independent energy source in communication with the high frequency sound wave generator. In another embodiment, the hearing loss alleviating device may further comprise a frequency adjuster to vary the wavelength of the high frequency sound being emitted from the high frequency sound wave generator. In another embodiment, the hearing loss alleviating device may further comprise a timing circuit configured to shut down the high frequency sound wave generator after a predetermined amount of elapsed time. In another embodiment, the hearing loss alleviating device may further comprise a control configured to adjust intensity of the high frequency sound waves emitted by the high frequency sound wave generator.

In another embodiment, the hearing loss alleviating device may further comprise a conduit, the conduit may include: a high frequency sound wave delivery system in acoustic communication with the high frequency sound wave generator; and an acoustic lens at a distal end of the conduit, the acoustic lens in communication with a display system.

In another embodiment, there is provided a hearing loss alleviating device sized and configured to be received, at least in part, by a central auditory system of a subject, the device comprising: a power supply; an electromagnetic radiation generator configured to irradiate at least a part of the central auditory system of the subject; an interface configured to selectably adjust electromagnetic radiation in a wavelength range of 10 nm-1 mm produced by the electromagnetic radiation generator when energized by the power supply; an electromagnetic field generator configured to produce an electromagnetic field within at least a part of the central auditory system of the subject; and an interface configured to selectably adjust the electromagnetic field in a range of 0.001-200 μT produced by the electromagnetic field generator when energized by the power supply.

In another embodiment, there is provided a hearing loss alleviating device sized and configured to be received, at least in part, by a central auditory system of a subject, the device comprising: a power supply; an electromagnetic radiation generator configured to irradiate at least a part of the central auditory system of the subject; an interface configured to selectably adjust electromagnetic radiation in a wavelength range of 10 nm-1 mm produced by the electromagnetic radiation generator when energized by the power supply; a high frequency sound wave generator configured to emit high frequency sound waves into at least a part of the central auditory system of the subject; and an interface configured to selectably adjust high frequency sound waves in a frequency range of 800,000 Hz-2,000,000 Hz produced by the high frequency sound wave generator when energized by the power supply.

In another embodiment, there is provided a hearing loss alleviating device sized and configured to be received, at least in part, by a central auditory system of a subject, the device comprising: a power supply; an electromagnetic field generator configured to produce an electromagnetic field within at least a part of the central auditory system of the subject; an interface configured to selectably adjust the electromagnetic field in a range of 0.001-200 μT produced by the electromagnetic field generator when energized by the power supply; a high frequency sound wave generator configured to emit high frequency sound waves into at least a part of the central auditory system of the subject; and an interface configured to selectably adjust high frequency sound waves in a frequency range of 800,000 Hz-2,000,000 Hz produced by the high frequency sound wave generator when energized by the power supply.

In another embodiment, there is provided a hearing loss alleviating device sized and configured to be received, at least in part, by a central auditory system of a subject, the device comprising: a power supply; an electromagnetic radiation generator configured to irradiate at least a part of the central auditory system of the subject; an interface configured to selectably adjust electromagnetic radiation in a wavelength range of 10 nm-1 mm produced by the electromagnetic radiation generator when energized by the power supply; an electromagnetic field generator configured to produce an electromagnetic field within at least a part of the central auditory system of the subject; an interface configured to selectably adjust the electromagnetic field in a range of 0.001-200 μT produced by the electromagnetic field generator when energized by the power supply; a high frequency sound wave generator configured to emit high frequency sound waves into at least a part of the central auditory system of the subject; and an interface configured to selectably adjust high frequency sound waves in a frequency range of 800,000 Hz-2,000,000 Hz produced by the high frequency sound wave generator when energized by the power supply.

In another embodiment, there is provided a method of treating or ameliorating hearing loss, the hearing loss having an aetiology selected from the group consisting of: (a) impaired blood flow; (b) impaired metabolism; (c) neuronal damage; (d) infection; and any combination of (a) to (d); the method comprising: irradiating at least a part of a central auditory system of a subject having hearing loss with electromagnetic radiation having a wavelength in a range of 10 nm-1 mm to stimulate an alleviating effect on the hearing loss of the subject; and exposing at least a part of a central auditory system of a subject having hearing loss to an electromagnetic field having an electromagnetic field strength in a range of 0.001-200 μT to stimulate an alleviating effect on the hearing loss of the subject.

In another embodiment, there is provided a method of treating or ameliorating hearing loss, the hearing loss having an aetiology selected from the group consisting of: (a) impaired blood flow; (b) impaired metabolism; (c) neuronal damage; (d) infection; and any combination of (a) to (d); the method comprising: irradiating at least a part of a central auditory system of a subject having hearing loss with electromagnetic radiation having a wavelength in a range of 10 nm-1 mm to stimulate an alleviating effect on the hearing loss of the subject; and exposing at least a part of a central auditory system of a subject having hearing loss to high frequency sound waves in a frequency range of 800,000 Hz-2,000,000 Hz to stimulate an alleviating effect on the hearing loss of the subject.

In another embodiment, there is provided a method of treating or ameliorating hearing loss, the hearing loss having an aetiology selected from the group consisting of: (a) impaired blood flow; (b) impaired metabolism; (c) neuronal damage; (d) infection; and any combination of (a) to (d); the method comprising: exposing at least a part of a central auditory system of a subject having hearing loss to an electromagnetic field having an electromagnetic field strength in a range of 0.001-200 μT to stimulate an alleviating effect on the hearing loss of the subject; and exposing at least a part of a central auditory system of a subject having hearing loss to high frequency sound waves in a frequency range of 800,000 Hz-2,000,000 Hz to stimulate an alleviating effect on the hearing loss of the subject.

In another embodiment, there is provided a method of treating or ameliorating hearing loss, the hearing loss having an aetiology selected from the group consisting of: (a) impaired blood flow; (b) impaired metabolism; (c) neuronal damage; (d) infection; and any combination of (a) to (d); the method comprising: irradiating at least a part of a central auditory system of a subject having hearing loss with electromagnetic radiation having a wavelength in a range of 10 nm-1 mm to stimulate an alleviating effect on the hearing loss of the subject; exposing at least a part of a central auditory system of a subject having hearing loss to an electromagnetic field having an electromagnetic field strength in a range of 0.001-200 μT to stimulate an alleviating effect on the hearing loss of the subject; and exposing at least a part of a central auditory system of a subject having hearing loss to high frequency sound waves in a frequency range of 800,000 Hz-2,000,000 Hz to stimulate an alleviating effect on the hearing loss of the subject.

In another embodiment, the subject may be a mammal. In another embodiment, the mammal may be a human.

Unless the meaning is clearly to the contrary, all ranges set forth herein are deemed inclusive of the endpoints.

An embodiment of the invention is now described, by way of example, with reference to the accompanying drawing.

### BRIEF DESCRIPTION OF THE DRAWING

FIG. 1 shows a perspective view of a hearing loss alleviating device in accordance with one embodiment of the invention.

FIG. 2 shows a front view of a hearing loss alleviating device in use in accordance with one embodiment of the invention.

FIG. 3 shows one perspective view of a hearing loss alleviating device in use in accordance with one embodiment of the invention.

FIG. 4 shows another perspective view of a hearing loss alleviating device in use in accordance with one embodiment of the invention.

### DETAILED DESCRIPTION

The present disclosure is directed, at least in part, to a hearing loss alleviating device sized and configured to be received, at least in part, by a central auditory system of a subject. The present disclosure also encompasses methods of treating or ameliorating hearing loss. The aetiology of the hearing loss so treated or ameliorated may be selected from impaired blood flow, impaired metabolism, neuronal damage, infection; and any combination of the afore-mentioned aetiologies.

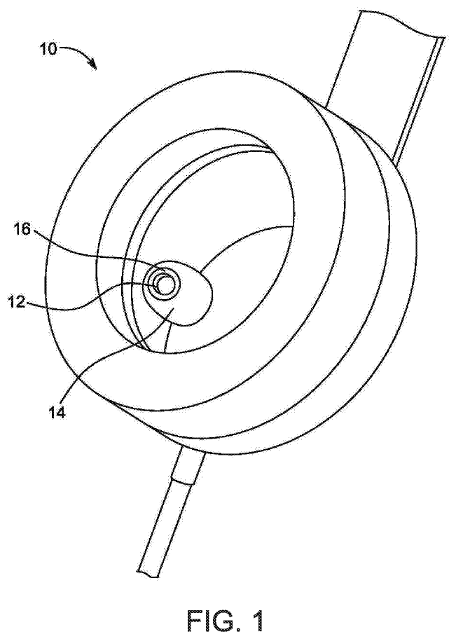

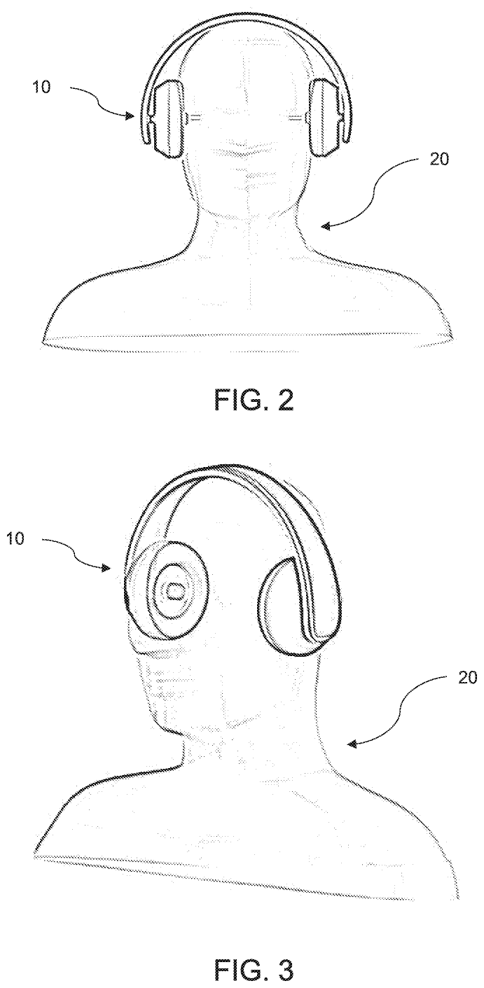

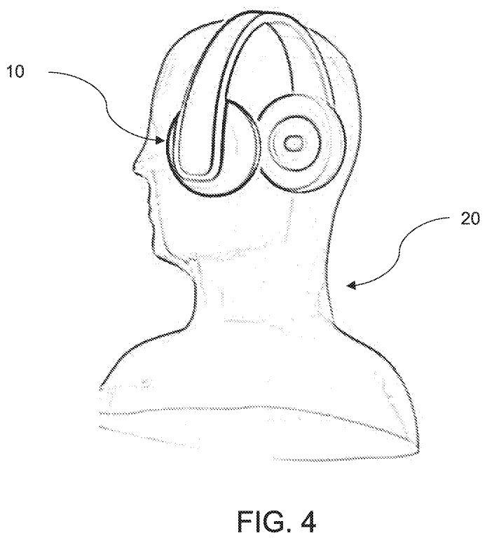

Referring to FIGS. 1 to 4, reference numeral 10 generally refers to an embodiment of a hearing loss alleviating device sized and configured to be received, at least in part, by a central auditory system of a human 20 (schematically shown in FIGS. 2 to 4). The hearing loss alleviating device 10 comprises an electromagnetic radiation generator (not shown) configured to irradiate at least a part of the central auditory system of the human and a power supply (not shown). The hearing loss alleviating device 10 also comprises an interface (not shown) configured to selectably adjust electromagnetic radiation in a wavelength range of 10 nm-1 mm produced by the electromagnetic radiation generator when energized by the power supply. The hearing loss alleviating device 10 comprises a speaker 12 in electrical communication with the power supply. The speaker 12 is configured to electrically communicate with a speaker controller (not shown). The speaker 12 is housed in a housing 14 and the housing is sized and configured to be received by an ear canal of the human. The speaker housing 14 have arranged thereon an electromagnetic radiation source 16. The electromagnetic radiation source 16 is in electrical communication with the power supply, the electromagnetic radiation generator and an electromagnetic radiation source controller (not shown).

It will be appreciated that the choice of wavelength of the electromagnetic radiation may be dependent, at least in part, on the depth of the targeted treatment area. Generally, although not exclusively, shorter wavelengths of less than or equal to about 700 nanometres (nm) are used to treat tissue proximal to the electromagnetic radiation source whereas wavelengths above about 700 nm penetrate further and are typically used to treat tissue distal to the electromagnetic radiation source. It will be appreciated that typically, although not exclusively, electromagnetic radiation of about 400 nm to about 470 nm has depth of electromagnetic radiation penetration of <1 mm; electromagnetic radiation between about 475 nm to about 545 nm has depth of 0.3 to 0.5 mm; electromagnetic radiation of about 570 nm to about 590 nm has a depth of 0.5 mm to 2 mm; electromagnetic radiation of about 630 nm to about 790 nm has depth of 2 to 3 mm and 800 nm to about 1.2 μm has a depth of 5 mm to 10 mm. In general, preferred embodiments, the wavelength may be in the range between about 10 nm to about 1 mm. Preferably, the electromagnetic radiation is in the visible range of the electromagnetic spectrum. In preferred embodiments, the wavelength of the electromagnetic radiation source is between about 400 nm and about 1 mm, more preferably about 630 nm to about 1 mm, even more preferably about 630 nm to about 790 nm. In other preferred embodiments about 800 nm to about 1 mm. In preferred embodiments, a wavelength is selected from the group consisting of about 630 nm, about 653 nm, about 660 nm, about 750 nm and about 830 nm, and any combination thereof. Ranges are to be interpreted as being fully inclusive of all values between the limits.

The present disclosure contemplates hearing loss alleviating devices and methods to irradiate a central auditory system in a mammal, preferably a human, that uses a single wavelength (monotherapy) as well irradiating a target area with a combination of different wavelengths (so-called “combination treatment”). Combination treatment may be useful to target various depths of tissue within a central auditory system. Although the wavelengths are different in a combination treatment modality, other parameters such as dose, fluence, power density, pulse structure and timing may be the same or different. Such parameters will depend on the application, as will be known by a person of skill in the art. By way of example, a combination treatment may comprise a wavelength from the red spectra and a wavelength from the near infra-red at the same or different fluence, dose, power density, pulse structure and timing. A treatment regime may include a single exposure, alternatively a treatment course of over a number of hours, days or weeks, as is required.

The electromagnetic radiation generator contemplated for use in the present invention is configured to emit electromagnetic radiation at one or more desired wavelengths. It will be appreciated that the present disclosure envisages inclusion of one type of electromagnetic radiation generator in the hearing loss alleviating device, alternatively a combination of electromagnetic radiation generators. Preferably, the electromagnetic radiation generator is selected from the group consisting of a light emitting diode, a laser, an arc lamp, an incandescent lamp, or any combination of the afore-mentioned light sources. In certain preferred embodiments, the electromagnetic radiation generator uses photons at a non-thermal irradiance to alter a biological activity, otherwise termed ‘low level light therapy’. In other preferred embodiments, the electromagnetic radiation source is a coherent light source (laser) or a non-coherent light source such as a filtered lamp or a light emitting diode (LED), or a combination of a coherent light source and a non-coherent light source. In suitable embodiments that relate to low level light therapy, the electromagnetic radiation source is a coherent light source or a non-coherent light source, or a combination thereof. According to the embodiments relating to low level light therapy, the non-coherent light source is a filtered lamp or a light emitting diode, or a combination thereof.

In preferred embodiments, the electromagnetic radiation generator includes at least one light emitting diode (LED), as is known in the art. The LED device may be configured to emit one or a plurality of wavelengths or spectral ranges. By way of example, an LED device may be a red LED that emits only in the red spectral range. Alternatively, the LED device may be configured to emit electromagnetic radiation in the red spectral range and the near infra-red spectral range. In some preferred embodiments, the LED is an organic LED (‘OLED’). An OLED, as would be known by a skilled addressee, emits light due to electroluminescence of thin films of organic semiconductors. An OLED comprises a light emitting organic material that is suitable for use in an OLED. Preferably, the light emitting organic material is selected from a small molecule, a polymer, a dendrimer and any combinations thereof. It will be appreciated that an OLED may have a plurality of layers comprising a light emitting organic material. In an OLED, the organic semiconductors may be deposited on a glass substrate or on substrate constructed from a flexible plastic such as, but not limited to, polyethylene terephthalate. A flexible OLED offers particular advantages of being bendable and lightweight. The invention also contemplates use of an inorganic LED. Preferably, the inorganic LED is a flexible LED. The garment of the invention contemplates preferred embodiments a plurality of LEDs comprising at least one inorganic LED and at least one organic LED. Other embodiments relating to an LED contemplate a quantum dot LED.

Various laser or non-coherent light sources are suitable for use in the present invention including inert gas lasers and semiconductor laser diodes such as Helium Neon (HeNe; about 633 nm), Ruby (about 694 nm), Argon (about 488 nm and about 514 nm), Krypton (about 521, about 530, about 568, about 647 nm), Gallium Arsenide (GaAs; >about 760 nm, with a common example of about 904 nm), and Gallium Aluminium Arsenide (GaAlAs; about 612 to about 870 nm). Ranges are to be interpreted as being fully inclusive of all values between the limits.

Delivery of the electromagnetic radiation and in particular delivery of light by an LED device may be either continuous or in a pulsed mode with specific pulse sequences and durations. The choice of either continuous mode or pulsed mode is dependent on the application as is known by a skilled addressee. It is also contemplated that certain embodiments employ a combination of a continuous mode and a pulsed mode.

Any suitable power source may be included in the hearing loss alleviating device of the present disclosure. The power source may be a full battery unit integral with the hearing loss alleviating device. Alternatively, the power source may be configured to be rechargeable, for example by inclusion of an AC power socket or a USB socket. It will be appreciated that the power source may be configured to suit a particular electromagnetic radiation generator, as will be known by a person of skill in the art.

Preferred embodiments relating to the hearing loss alleviating device of the present disclosure include a timing circuit configured to stop the generation of electromagnetic radiation after a predetermined amount of elapsed time. Such a timing circuit is particularly advantageous for a pulsed mode of operation. For example, a predetermined amount of elapsed period of time may be about 10 seconds, about 20 seconds, about 30 seconds, about 40 seconds, about 50 seconds, and about 60 seconds or more, 1 hour or 2 hours. The predetermined amount of elapsed time is dependent on the application/treatment envisaged.

The present disclosure contemplates embodiments that include a frequency adjuster to adjust a wavelength of electromagnetic radiation emitted from the electromagnetic radiation generator. This is advantageous if a plurality of wavelengths is desired. By way of example, the hearing loss alleviating device may include a red LED device configured to emit light at any wavelength in the red spectral range and an adjuster for adjusting to a wavelength between about 630 nm and about 700 nm accordingly. A conventional adjuster is contemplated and may include a processor.

The present disclosure contemplates hearing loss alleviating devices and methods to produce an electromagnetic field in a range of 0.001-200 μT within at least a part of the central auditory system of a subject. Preferred embodiments may comprise wire coils for producing a magnetic field. In some embodiments, the induced magnetic fields are oscillating magnetic fields, such as a 50 Hertz pulsed field. A person skilled in the art would be aware that the range frequencies of the pulse field may include, but not be limited too, 10-100 Hertz pulsed fields.

The present disclosure contemplates hearing loss alleviating devices and methods to produce high frequency sound waves in a frequency range of 800,000 Hz-2,000,000 Hz within at least a part of the central auditory system of a subject. A person skilled in the art would be aware that the range frequencies of the pulse field may include pulsed emission of the high frequency sound waves selected from the frequency range of 800,000 Hz-2,000,000 Hz.

The present disclosure contemplates methods of treating or ameliorating hearing loss as described herein.

The methods of the disclosure discussed herein include methods that include treating or ameliorating hearing loss associated with an aetiology selected from the group consisting of impaired blood flow, impaired metabolism, neuronal damage, infection; and any combination of the afore-mentioned aetiologies. The central auditory system as used herein is inclusive of tissues of the cochlea and divisions thereof, the vestibular system, the vestibulocochlear nerves, and the auditory systems in the brain per se.

In particular embodiments, methods of the invention include effects at the molecular, cellular, and tissue levels. In one aspect of the invention, the method of the invention includes stimulation of the metabolic flux of a cell. In particular, the stimulation may be associated with the flux through pathways such as glycolysis, the citric acid cycle, oxidative phosphorylation, β-oxidation, and anaplerotic reactions associated with the citric acid cycle.

The method of the invention may also be associated with the modulation of the transcription factors, nuclear receptors, and like proteins that modulate gene expression.

The method of the invention may also be associated with the modulation of the redox potential of cells. Such modulation of the redox potential may be linked with redox factor-1 (Ref-1) dependent activator protein-1 (AP-1)(a heterodimer of c-Fos and c-Jun), nuclear factor kappa B (NF-κB), p53, activating transcription factor/cAMP-response element-binding protein (ATF/CREB), hypoxia-inducible factor (HIF)-1, and HIF-like factor. Transcription factors may be associated with protein synthesis that initiates downstream effects such as increased cell proliferation and migration, modulation in the levels of cytokines, growth factors and inflammatory mediators, and increased tissue oxygenation.

The method may also be associated with a modulation of the levels of reactive oxygen species (ROS) within a cell.

The method may further be associated with the modulation of transporter proteins, neurotransmitters and/or neuromodulators in the central auditory systems. The neurotransmitters in a central auditory system modulated by the method of the invention may include both excitatory and inhibitory neurotransmitters.

Neurotransmission of particular relevance includes, but is not limited to, glutamatergic, glycinergic and GABAergic neurotransmission.

The method may further be associated with the modulation of the receptor associated auditory sensation, in particular glutamate receptors, GABA receptors and/or glycine receptors of the central auditory system.

The method may be of particular relevance in the invigoration of components of the cochlea, such as outer hair cells associated with cochlear amplification, inner hair cells associated with the transduction of nerve impulses via a glutamatergic connection with afferent dendritic nerve endings from spiral ganglion cells, and invigoration of axons of the auditory nerves extending via glutamatergic synapses from the cochlea to the ventral and dorsal cochlear nuclei, nuclei of the superior olivary complex and lateral lemniscus and the inferior colliculus, in particular the lateral and medial superior olive and the medial nucleus of the trapezoid body.

Methods of the disclosure include methods of treating or ameliorating hearing loss. As used herein, the terms “treatment,” “treating,” and the like, refer to obtaining a desired pharmacologic and/or physiologic effect. The effect may be therapeutic in terms of a partial or complete remediation of hearing loss.

It is well established that if the electromagnetic radiation applied is not of sufficient irradiance or the irradiation time is too short then there is no response. If the irradiance is too high or irradiation time is too long, then the response may be inhibited. Somewhere in between is the optimal combination of irradiance and time for stimulation. It will be appreciated that parameters such as duration of exposure (can be known as irradiation time), power of electromagnetic radiation (mW), energy (J) of electromagnetic radiation, suitable type of electromagnetic radiation source, number of treatments or exposures, treatment interval (hours, days or weeks), energy density (J/cm²), wavelength of electromagnetic radiation and depth of penetration of electromagnetic radiation is dependent, at least in part, upon the method being employed and as will be known by a person of skill in the art. Typically, although not exclusively, in those embodiments that contemplate low level electromagnetic radiation therapy, it uses relatively low fluences (between about 0.04 to about 50 J/cm²) and power densities (<100 mW/cm²). Ranges are to be interpreted as being fully inclusive of all values between the limits.

The following prophetic examples are made:

### Example 1

A treatment using the hearing loss alleviating device of the present disclosure will be irradiance (Intensity) at 1 milli watts/cm², the wavelengths that will be used are wavelength 1 of 653 nm and wavelength 2 of 750 nm for treatment duration of 20 minutes. The hearing loss alleviating device may be inserted into a central auditory system of a subject by means of a translabyrinthine approach. This approach will comprise an incision of a C shape behind the pinna of the central auditory system of a subject to undergo treatment. At least part of the mastoid bone is removed to provide access. This method permits ready access of the hearing loss alleviating device to the central auditory system. The hearing loss alleviating device is sized and configured to be at least partly resident in the central auditory system for the duration of the treatment, i.e. a fixed term, an extended term or permanent.

### Example 2

A treatment using the hearing loss alleviating device of the present disclosure will be irradiance (Intensity) at 1 milli watts/cm², the wavelengths that will be used are wavelength 1 of 653 nm and wavelength 2 of 750 nm for treatment duration of 20 minutes. The hearing loss alleviating device may be inserted into a central auditory system of a subject by means of a middle fossa approach. This approach will comprise an incision over the pinna and at least part of the temporal bone and a portion of the meninges in the vicinity of the central auditory system to be treated as appropriate will be removed to provide access to the central auditory system to be treated. This method permits ready access of the hearing loss alleviating device to the central auditory system. Where appropriate the hearing loss alleviating device is sized and configured to be at least partly resident in the central auditory system for the duration of the treatment, i.e., a fixed term, an extended term or permanent.

### Example 3

A treatment using the hearing loss alleviating device of the present disclosure will be irradiance (Intensity) at 1 milli watts/cm², the wavelengths that will be used are wavelength 1 of 653 nm and wavelength 2 of 750 nm for treatment duration of 20 minutes. The hearing loss alleviating device may be inserted into a central auditory system of a subject by means of a middle fossa approach. This approach will comprise an incision made behind the pinna and an opening in the skull created in the vicinity of the central auditory system to be treated to allow exposure of the central auditory system to be treated via the so-called retrosigmoid approach. This method permits ready access of the hearing loss alleviating device to the central auditory system. Where appropriate the hearing loss alleviating device is sized and configured to be at least partly resident in the central auditory system for the duration of the treatment. A person skilled in the art will be aware that this method is not be amenable to extended residencies for the hearing loss alleviating device in the absence of appropriate plating to protect vulnerable areas of the central nervous system.

### Example 4

A treatment using the hearing loss alleviating device of the present disclosure will be irradiance (Intensity) at 1 milli watts/cm², the wavelengths that will be used are wavelength 1 of 653 nm and wavelength 2 of 750 nm for treatment duration of 20 minutes. The hearing loss alleviating device may be inserted into a central auditory system of a subject by means of a translabyrinthine approach. This approach will comprise an incision of a C shape behind the pinna of the central auditory system of a subject to undergo treatment. At least part of the mastoid bone is removed to provide access. This method permits ready access of the hearing loss alleviating device to the central auditory system. The hearing loss alleviating device is sized and configured to be at least partly resident in the central auditory system for the duration of the treatment, i.e., a fixed term, an extended term or permanent. The hearing loss alleviating device further comprising at least one ear piece for insertion into an ear canal. The ear piece comprising a speaker for audio output. A skilled addressee will be aware that alternative approaches such as middle fossa approach, retrosigmoid approach, or like means of accessing a central auditory system may be employed in this example. In this example, a person testing a subject's hearing, e.g., an audiologist, may activate a sound signal to be output by the speaker to test the hearing of the subject who has the hearing loss alleviating device inserted into a central auditory system.

### Example 5

A treatment using the hearing loss alleviating device of the present disclosure will be irradiance (Intensity) at 1 milli watts/cm², the wavelengths that will be used are wavelength 1 of 653 nm and wavelength 2 of 750 nm for treatment duration of 20 minutes. The hearing loss alleviating device may be inserted into a central auditory system of a subject by means of a translabyrinthine approach. This approach will comprise an incision of a C shape behind the pinna of the central auditory system of a subject to undergo treatment. At least part of the mastoid bone is removed to provide access. This method permits ready access of the hearing loss alleviating device to the central auditory system. The hearing loss alleviating device is sized and configured to be at least partly resident in the central auditory system for the duration of the treatment, i.e., a fixed term, an extended term or permanent. The hearing loss alleviating device further comprising at least one ear piece for insertion into an ear canal. The ear piece comprising a speaker for audio output. A skilled addressee will be aware that alternative approaches such as middle fossa approach, retrosigmoid approach, or like means of accessing a central auditory system may be employed in this example. In this example, a subject may activate a sound signal to be output by the speaker while undergoing a treatment of a central auditory system.

### Example 6

A treatment using the hearing loss alleviating device of the present disclosure will be a 50 Hertz pulsed magnetic field of 100 μT. The hearing loss alleviating device may be inserted into a central auditory system of a subject by means of a translabyrinthine approach. This approach will comprise an incision of a C shape behind the pinna of the central auditory system of a subject to undergo treatment. At least part of the mastoid bone is removed to provide access. This method permits ready access of the hearing loss alleviating device to the central auditory system. The hearing loss alleviating device is sized and configured to be at least partly resident in the central auditory system for the duration of the treatment, i.e. a fixed term, an extended term or permanent.

### Example 7

A treatment using the hearing loss alleviating device of the present disclosure will be a 50 Hertz pulsed magnetic field of 100 μT. The hearing loss alleviating device may be inserted into a central auditory system of a subject by means of a middle fossa approach. This approach will comprise an incision over the pinna and at least part of the temporal bone and a portion of the meninges in the vicinity of the central auditory system to be treated as appropriate will be removed to provide access to the central auditory system to be treated. This method permits ready access of the hearing loss alleviating device to the central auditory system. Where appropriate the hearing loss alleviating device is sized and configured to be at least partly resident in the central auditory system for the duration of the treatment, i.e. a fixed term, an extended term or permanent.

### Example 8

A treatment using the hearing loss alleviating device of the present disclosure will be a 50 Hertz pulsed magnetic field of 100 μT. The hearing loss alleviating device may be inserted into a central auditory system of a subject by means of a middle fossa approach. This approach will comprise an incision made behind the pinna and an opening in the skull created in the vicinity of the central auditory system to be treated to allow exposure of the central auditory system to be treated via the so-called retrosigmoid approach. This method permits ready access of the hearing loss alleviating device to the central auditory system. Where appropriate the hearing loss alleviating device is sized and configured to be at least partly resident in the central auditory system for the duration of the treatment. A person skilled in the art will be aware that this method is not be amenable to extended residencies for the hearing loss alleviating device in the absence of appropriate plating to protect vulnerable areas of the central nervous system.

### Example 9

A treatment using the hearing loss alleviating device of the present disclosure will be a 50 Hertz pulsed magnetic field of 100 μT. The hearing loss alleviating device may be inserted into a central auditory system of a subject by means of a translabyrinthine approach. This approach will comprise an incision of a C shape behind the pinna of the central auditory system of a subject to undergo treatment. At least part of the mastoid bone is removed to provide access. This method permits ready access of the hearing loss alleviating device to the central auditory system. The hearing loss alleviating device is sized and configured to be at least partly resident in the central auditory system for the duration of the treatment, i.e. a fixed term, an extended term or permanent. The hearing loss alleviating device further comprising at least one ear piece for insertion into an ear canal. The ear piece comprising a speaker for audio output. A skilled addressee will be aware that alternative approaches such as middle fossa approach, retrosigmoid approach, or like means of accessing a central auditory system may be employed in this example. In this example, a person testing a subject's hearing, e.g., an audiologist, may activate a sound signal to be output by the speaker to test the hearing of the subject who has the hearing loss alleviating device inserted into a central auditory system.

### Example 10

A treatment using the hearing loss alleviating device of the present disclosure will be a 50 Hertz pulsed magnetic field of 100 μT. The hearing loss alleviating device may be inserted into a central auditory system of a subject by means of a translabyrinthine approach. This approach will comprise an incision of a C shape behind the pinna of the central auditory system of a subject to undergo treatment. At least part of the mastoid bone is removed to provide access. This method permits ready access of the hearing loss alleviating device to the central auditory system. The hearing loss alleviating device is sized and configured to be at least partly resident in the central auditory system for the duration of the treatment, i.e. a fixed term, an extended term or permanent. The hearing loss alleviating device further comprising at least one ear piece for insertion into an ear canal. The ear piece comprising a speaker for audio output. A skilled addressee will be aware that alternative approaches such as middle fossa approach, retrosigmoid approach, or like means of accessing a central auditory system may be employed in this example. In this example, a subject may activate a sound signal to be output by the speaker while undergoing a treatment of a central auditory system.

### Example 11

A set of earphones comprising the hearing loss alleviating device of the present disclosure will irradiate the ear canal of a user at selected wavelengths between 10 nm and 1 mm for a selectable time. The earphones will include at least one speaker for audio output so that a user can receive audio input while using the earphones.

### Example 12

A set of earphones comprising the hearing loss alleviating device of the present disclosure will irradiate the ear canal of a user at selected wavelengths between 10 nm and 1 mm for a selectable time. The measured dose delivered by the earphones may, for example, be 1 milliwatt per cm² for 5 minutes. The earphones will include at least one speaker for audio output so that a user can receive audio input while using the earphones. The audio input may, for example, be music.

### Example 13

A set of earphones comprising the hearing loss alleviating device of the present disclosure will irradiate the ear canal of a user at selected wavelengths between 10 nm and 1 mm for a selectable time. The measured dose delivered by the earphones may, for example, be 1 milliwatt per cm² for 5 minutes. The earphones will include at least one speaker for audio output so that a user can receive audio input while using the earphones. The audio input may, for example, be music. The electromagnetic radiation delivered to the ear canal may be distributed to tissue surrounding the ear canal by, for example, body fluid, blood, cartilage, bone or the like.

### Example 14

A set of earphones comprising the hearing loss alleviating device of the present disclosure will irradiate the ear canal of a user at selected wavelengths between 10 nm and 1 mm for a selectable time. The measured dose delivered by the earphones may, for example, be 1 milliwatt per cm² for 5 minutes. The earphones will include at least one speaker for audio output so that a user can receive audio input while using the earphones. The audio input may, for example, be music. The electromagnetic radiation delivered to the ear canal may be distributed to tissue surrounding the ear canal by, for example, body fluid, blood, cartilage, bone or the like. The earphones may accommodate and enclose the outer ear(s) of the user.

### Example 15

A set of earphones comprising the hearing loss alleviating device of the present disclosure will irradiate the ear canal of a user at selected wavelengths between 10 nm and 1 mm for a selectable time. The measured dose delivered by the earphones may, for example, be 1 milliwatt per cm² for 5 minutes. The earphones will include at least one speaker for audio output so that a user can receive audio input while using the earphones. The audio input may, for example, be music. The electromagnetic radiation delivered to the ear canal may be distributed to tissue surrounding the ear canal by, for example, body fluid, blood, cartilage, bone or the like. The earphones may accommodate and enclose the outer ear(s) of the user. The earphones may be supported by the area surrounding the ear, i.e., overlying the temporal and/or mastoid bones.

The set of earphones of examples 10 to 15 may include a magnetic field generator to generate a magnetic field in the inner ear and surrounding tissues of a user.

A person skilled in the art will appreciate that the electromagnetic radiation emitted by the device of the examples may be transmitted by tissue such as skin, bone, cartilage, body fluids into a central auditory system of a subject.

### Example 16

A treatment using the hearing loss alleviating device of the present disclosure will be high frequency sound waves, the frequency that will be used is 1 MHz for treatment duration of 20 minutes. The hearing loss alleviating device may be inserted into a central auditory system of a subject by means of a translabyrinthine approach. This approach will comprise an incision of a C shape behind the pinna of the central auditory system of a subject to undergo treatment. At least part of the mastoid bone is removed to provide access. This method permits ready access of the hearing loss alleviating device to the central auditory system. The hearing loss alleviating device is sized and configured to be at least partly resident in the central auditory system for the duration of the treatment, i.e. a fixed term, an extended term or permanent.

### Example 17

A treatment using the hearing loss alleviating device of the present disclosure will be high frequency sound waves, the frequency that will be used is 1 MHz for treatment duration of 20 minutes. The hearing loss alleviating device may be inserted into a central auditory system of a subject by means of a middle fossa approach. This approach will comprise an incision over the pinna and at least part of the temporal bone and a portion of the meninges in the vicinity of the central auditory system to be treated as appropriate will be removed to provide access to the central auditory system to be treated. This method permits ready access of the hearing loss alleviating device to the central auditory system. Where appropriate the hearing loss alleviating device is sized and configured to be at least partly resident in the central auditory system for the duration of the treatment, i.e., a fixed term, an extended term or permanent.

### Example 18

A treatment using the hearing loss alleviating device of the present disclosure will be high frequency sound waves, the frequency that will be used is 1 MHz for treatment duration of 20 minutes. The hearing loss alleviating device may be inserted into a central auditory system of a subject by means of a middle fossa approach. This approach will comprise an incision made behind the pinna and an opening in the skull created in the vicinity of the central auditory system to be treated to allow exposure of the central auditory system to be treated via the so-called retrosigmoid approach. This method permits ready access of the hearing loss alleviating device to the central auditory system. Where appropriate the hearing loss alleviating device is sized and configured to be at least partly resident in the central auditory system for the duration of the treatment. A person skilled in the art will be aware that this method is not be amenable to extended residencies for the hearing loss alleviating device in the absence of appropriate plating to protect vulnerable areas of the central nervous system.

### Example 19

A treatment using the hearing loss alleviating device of the present disclosure will be high frequency sound waves, the frequency that will be used is 1 MHz for treatment duration of 20 minutes. The hearing loss alleviating device may be inserted into a central auditory system of a subject by means of a translabyrinthine approach. This approach will comprise an incision of a C shape behind the pinna of the central auditory system of a subject to undergo treatment. At least part of the mastoid bone is removed to provide access. This method permits ready access of the hearing loss alleviating device to the central auditory system. The hearing loss alleviating device is sized and configured to be at least partly resident in the central auditory system for the duration of the treatment, i.e., a fixed term, an extended term or permanent. The hearing loss alleviating device further comprising at least one ear piece for insertion into an ear canal. The ear piece comprising a speaker for audio output. A skilled addressee will be aware that alternative approaches such as middle fossa approach, retrosigmoid approach, or like means of accessing a central auditory system may be employed in this example. In this example, a person testing a subject's hearing, e.g., an audiologist, may activate a sound signal to be output by the speaker to test the hearing of the subject who has the hearing loss alleviating device inserted into a central auditory system.

### Example 20

A treatment using the hearing loss alleviating device of the present disclosure will be high frequency sound waves, the frequency that will be used is 1 MHz for treatment duration of 20 minutes. The hearing loss alleviating device may be inserted into a central auditory system of a subject by means of a translabyrinthine approach. This approach will comprise an incision of a C shape behind the pinna of the central auditory system of a subject to undergo treatment. At least part of the mastoid bone is removed to provide access. This method permits ready access of the hearing loss alleviating device to the central auditory system. The hearing loss alleviating device is sized and configured to be at least partly resident in the central auditory system for the duration of the treatment, i.e., a fixed term, an extended term or permanent. The hearing loss alleviating device further comprising at least one ear piece for insertion into an ear canal. The ear piece comprising a speaker for audio output. A skilled addressee will be aware that alternative approaches such as middle fossa approach, retrosigmoid approach, or like means of accessing a central auditory system may be employed in this example. In this example, a subject may activate a sound signal to be output by the speaker while undergoing a treatment of a central auditory system.

### Example 21

A set of earphones comprising the hearing loss alleviating device of the present disclosure will irradiate the ear canal of a user at selected wavelengths between 10 nm and 1 mm for a selectable time. The measured dose delivered by the earphones may, for example, be 1 milliwatt per cm² for 5 minutes. The earphones will include at least one speaker for audio output so that a user can receive audio input while using the earphones. The audio input may, for example, be music. The electromagnetic radiation delivered to the ear canal may be distributed to tissue surrounding the ear canal by, for example, body fluid, blood, cartilage, bone or the like. The earphones may accommodate and enclose the outer ear(s) of the user. The earphones may be supported by the area surrounding the ear, i.e., overlying the temporal and/or mastoid bones.

The set of earphones of example 21 may include a high frequency sound wave generator to generate high frequency sound waves in the inner ear and surrounding tissues of a user.

A person skilled in the art will appreciate that the high frequency sound waves emitted by the device of the examples may be transmitted by tissue such as skin, bone, cartilage, body fluids into a central auditory system of a subject.

It is to be understood that the terminology employed above is for the purpose of description and should not be regarded as limiting. The described embodiments are intended to be illustrative of the invention, without limiting the scope thereof. The invention is capable of being practised with various modifications and additions as will readily occur to those skilled in the art.

1. A hearing loss alleviating device sized and configured to be received, at least in part, by a central auditory system of a subject, the device comprising:
an electromagnetic radiation generator configured to irradiate at least a part of the central auditory system of the subject;
a power supply; and
an interface configured to selectably adjust electromagnetic radiation in a wavelength range of 10 nm-1 mm produced by the electromagnetic radiation generator when energized by the power supply.

2. The hearing loss alleviating device of claim 1, including an electromagnetic radiation generator controller in communication with the electromagnetic radiation generator.

3. The hearing loss alleviating device of claim 1, including an independent energy source in communication with the electromagnetic radiation generator.

4. The hearing loss alleviating device of claim 1, wherein the electromagnetic radiation generator includes at least one light emitting diode.

5. The hearing loss alleviating device of claim 1, wherein the electromagnetic radiation generator is a laser.

6. The hearing loss alleviating device of claim 1, wherein the electromagnetic radiation generator is an arc lamp.

7. The hearing loss alleviating device of claim 1, wherein the electromagnetic radiation generator is an incandescent lamp.

8. The hearing loss alleviating device of claim 1, further comprising a frequency adjuster to vary the wavelength of the electromagnetic radiation being emitted from the electromagnetic radiation generator.

9. The hearing loss alleviating device of claim 1, further comprising a timing circuit configured to shut down the electromagnetic radiation generator after a predetermined amount of elapsed time.

10. The hearing loss alleviating device of claim 1, further comprising a control configured to adjust intensity of the electromagnetic radiation emitted by the electromagnetic radiation generator.

11. The hearing loss alleviating device of claim 1, further comprising a conduit, the conduit including:
an electromagnetic radiation delivery system in optical communication with the electromagnetic radiation generator; and an objective lens at a distal end of the conduit, the objective lens in communication with a display system.

12-21. (canceled)

22. The hearing loss alleviating device of claim 1, further comprising a speaker.

23. The hearing loss alleviating device of claim 22, wherein the speaker is in electrical communication with the power supply.

24. (canceled)

25. The hearing loss alleviating device of claim 22, wherein the speaker is housed in a housing and the housing is sized and configured to be received by an ear canal of a subject.

26. The hearing loss alleviating device of claim 25, wherein the housing has arranged thereon at least one electromagnetic radiation source.

27. The hearing loss alleviating device of claim 26, wherein the at least one electromagnetic radiation source is in electrical communication with the power supply.

28. The hearing loss alleviating device of claim 26, wherein the at least one electromagnetic radiation source is in operative communication with the electromagnetic radiation generator.

29. The hearing loss alleviating device claim 26, further comprising an electromagnetic radiation source controller in electrical communication with the at least one electromagnetic radiation source.

30-49. (canceled)

50. A hearing loss alleviating device sized and configured to be received, at least in part, by a central auditory system of a subject, the device comprising:
a power supply;
an electromagnetic radiation generator configured to irradiate at least a part of the central auditory system of the subject;
an interface configured to selectably adjust electromagnetic radiation in a wavelength range of 10 nm-1 mm produced by the electromagnetic radiation generator when energized by the power supply;
an electromagnetic field generator configured to produce an electromagnetic field within at least a part of the central auditory system of the subject;
an interface configured to selectably adjust the electromagnetic field in a range of 0.001-200 μT produced by the electromagnetic field generator when energized by the power supply;
a high frequency sound wave generator configured to emit high frequency sound waves into at least a part of the central auditory system of the subject; and an interface configured to selectably adjust high frequency sound waves in a frequency range of 800,000 Hz-2,000,000 Hz produced by the high frequency sound wave generator when energized by the power supply.

51-53. (canceled)

54. A method of treating or ameliorating hearing loss, the hearing loss having an aetiology selected from the group consisting of: (a) impaired blood flow; (b) impaired metabolism; (c) neuronal damage; (d) infection; and any combination of (a) to (d); the method comprising:
irradiating at least a part of a central auditory system of a subject having hearing loss with electromagnetic radiation having a wavelength in a range of 10 nm-1 mm to stimulate an alleviating effect on the hearing loss of the subject;
exposing at least a part of a central auditory system of a subject having hearing loss to an electromagnetic field having an electromagnetic field strength in a range of 0.001-200 μT to stimulate an alleviating effect on the hearing loss of the subject; and
exposing at least a part of a central auditory system of a subject having hearing loss to high frequency sound waves in a frequency range of 800,000 Hz-2,000,000 Hz to stimulate an alleviating effect on the hearing loss of the subject.
